(19) United States
(12) Patent Application Publication
(10) Pub. No.: US 2018/0168955 A1
(43) Pub. Date: Jun. 21, 2018

(54) IODINE-CONTAINING ORAL CARE COMPOSITIONS

(71) Applicant: Drew Shabo, Ooltewah, TN (US)

(72) Inventor: Drew Shabo, Ooltewah, TN (US)

(21) Appl. No.: 15/385,197

(22) Filed: Dec. 20, 2016

(51) Int. Cl. A61K 8/20; A61Q 11/00

(52) U.S. Cl. CPC A61K 8/20; A61K 2800/92; A61Q 11/00

(57) ABSTRACT

An oral composition that includes an oral care carrier for oral delivery and iodine salt, and a method of treatment of the oral cavity by orally administering the oral care composition to a subject in need thereof in order to enhance the health of the oral cavity and prevent and/or treat tooth decay and gum disease associated with, among other things, bacteria. The iodine salt that is present in the oral composition is preferably potassium iodide.

### BACKGROUND OF THE INVENTION

### Field of the Invention

The present invention relates to oral hygiene compositions and, more particularly, to oral hygiene compositions containing iodine salt for oral care, and to a method of using the oral hygiene compositions.

### Description of the Background Art

Various oral hygiene care products are currently in use. The most commonly used products are toothpaste, tooth gels and mouthwashes. Typically such products consist of a mild abrasive dispersed in a gel, paste base, or other soluble material with detergents added to aid in cleaning and tooth decay reduction.

The oral care industry has looked for many years for a way to prevent the growth and colonization of bacteria on teeth because adhered bacteria leads to formation of plaque, calculus, and ultimately, tooth-decay. As people live longer, dental care plays a larger role in the overall health.

Dental plaque results when cariogenic bacteria form deposits on tooth surfaces. The presence of the bacteria and deposits is detrimental to the health of the tooth for, if left unchecked, they may result in infected gingival tissue, the formation of dental caries and possibly periodontal disease. In extreme cases, bacteria may result in the loss of teeth.

It is generally recognized that a reduction in dental plaque promotes clean teeth, fresh breath, and healthy gums. The dental plaque, however, is very resistant to antimicrobial agents.

A reduction of the bacterial activity in the oral cavity is most important, as this activity is a major cause of most problems related to oral hygienic conditions. Antimicrobials agents that have been shown to have definite plaque-reducing abilities include chlorhexidine, cetylpyridinium chloride (CPC), Triclosan and Delmopinol. Essential oils such as thymol, Eucalyptol, methyl salicylate, and menthol along with other essential oils in an alcohol-based vehicle have also been found to reduce plaque. Generally, these oils benefit from the presence of an alcohol to facilitate their solubility and penetration of the plaque biofilm. While suitable for oral treatments, such as mouthwashes, high concentrations of alcohols can leave a bitter aftertaste in oral compositions, such as gums, edible films, and confectioneries, and the like.

Other attempts have been made to control or prevent both the occurrence of dental caries and the formation of dental plaque through treatment with antibacterial materials in a dental office at periodic, but not frequent, intervals. Such treatments are primarily intended to render tooth enamel more resistant to the acid action caused by plaque. They do not, however, result in plaque control for an extended period since plaque reestablishes itself on the teeth shortly after ingestion of food.

Additionally, fluoride solutions or gels have been known and widely used for oral hygiene product that are used to combat gum disease and tooth decay, such as stannous fluoride, which provides both fluoride ions for effectiveness in prevention of tooth decay and stannous ions for effectiveness in anti-plaque, anti-gingivitis, and hypersensitivity. While providing a number of benefits, however, stannous fluoride has a bitter salty taste and is also only slightly soluble in water, and accordingly does not readily dissociate to provide stannous ions.

Stannous fluoride is beneficially dissociated at a generally acidic pH. However, such a pH frustrates stability with other agents used in such compositions, such as the triclosan. Another significant problem that has been encountered in incorporating fluoride ions, through the use of fluorine-containing compounds, into toothpastes or other oral treatment compositions has been the fact that the abrasive or polishing ingredients of the composition react with the fluorine-containing inorganic compounds, and gradually, over a period of time, inactivate the fluorine-containing compounds by forming insoluble compounds, which do not ionize to release fluoride ions. Such insoluble compounds have no caries reducing or inhibiting properties. Hence, they do not bring about desired enamel hardening effects on the tooth enamel.

Further, although safe at low concentrations, sustained exposure to large quantities of soluble fluoride salts is dangerous. A case of fluoride poisoning was defined as an illness consisting of nausea, vomiting, diarrhea, abdominal pain, or numbness or tingling of the face or extremities. Chronic exposure to fluoride in large amounts may also interfere with bone formation. Consumption of fluoride at levels beyond those used in fluoridated water for a long period of time causes skeletal fluorosis.

The results of research support the possibility of an adverse effect of high fluoride exposure on children's neurodevelopment. All in all, overexposure to fluoride can lead to various serious health issues with respect to brain development, bone formation, kidney and thyroid functions, and teeth development.

Prolonged fluoridation is also known to result in dental fluorosis. Dental fluorosis may result in abnormal children's tooth development, and subsequent bone fracture problems and other possible adverse effects.

Accordingly, the need exists for an active ingredient, or a combination of active ingredients, other than fluoride solutions, that can provide the benefits of removing plaque, preventing or slowing down plaque formation and promoting an overall healthy oral cavity.

Iodine has been used to destroy viruses. For example, U.S. Pat. No. 4,355,021 discloses a substantially dry, impregnated wipe having iodine and a means for retaining the iodine. The iodine is present in the wipe in an amount from about 1% to about 15% by weight of the wipe and in an amount sufficient to provide virucidal activity. The iodine is preferably present in an amount of from about 2% to about 5%. A flexible paper is preferred, and the most preferred substrate is facial tissue. The cited patent, however, does not disclose an oral hygiene composition containing iodine.

Iodine is a halogen element of a peculiar odor and acrid taste. Iodine is characterized by the chemical symbol I, has an atomic number of 53, and an atomic weight of 126.904. It is a nonmetallic element, occurring in heavy, grayish black plates or granules. Iodine is essential in nutrition, being especially abundant in the colloid of the thyroid gland. One gram of iodine dissolves in 2950 ml of water or in 12.5 ml alcohol. Internally, iodine is used for treatment of goiter, hyperthyroidism and as an antidote to alkaloid poisons. Topically, iodine has been used as an antiseptic.

As discussed above, bacteria may enter the body through the mouth and adhere to teeth leading to formation of plaque, calculus, and ultimately, tooth-decay. Mouthwash, toothpastes and other gel solutions containing antimicrobial agents have been used to reduce or eliminate bacteria in the mouth and throat. For example, U.S. Pat. No. 4,959,204 discloses oral compositions such as toothpastes, mouthwashes, lozenges and chewing gum containing an antimicrobial agent which is effective against plaque/gingivitis and mouth odor. However, an iodine-containing mouthwash or toothpaste solutions for treating and/or enhancing health of the oral cavity are not known.

Another benefit of the solution containing iodine, such as potassium iodide, is that potassium iodide has shown marked ability to increase mental acuity.

It is, therefore, an object of this invention to disclose iodine-containing oral hygiene compositions, including, but not limited to mouthwash, toothpaste, dental cleaner, dental pastes, and denture cleaners. The iodine-containing solutions are useful for maintenance of the oral cavity and the prevention and/or treatment of tooth decay and gum disease associated with the bacteria, as well as providing for increase mental acuity.

It is a further object of this invention to disclose a method of use for the iodine-containing oral hygiene compositions, where the solutions are used for enhancing the health of the oral cavity and preventing and/or treating tooth decay and gum disease associated, among other things, with the bacteria.

### SUMMARY OF THE INVENTION

The present invention discloses an oral composition that includes an oral care carrier for the oral delivery and iodine salt, and a method of treatment of an oral cavity by orally administering the oral care composition to a subject in need thereof for enhancing the health of the oral cavity and preventing and/or treating tooth decay and gum disease.

The iodine salt is present in the oral composition in a concentration range of 0.05%-25.0%.

The iodine salt may be selected from the group consisting of ammonium iodate, ammonium iodide, calcium iodate, calcium iodide, iodine monochloride, iodine trichloride, magnesium iodate, magnesium iodide, potassium iodate, potassium iodide, sodium iodate, sodium iodide, zinc iodate and zinc iodide.

In one preferred embodiment of this disclosure, the iodine salt that is present in the oral composition is potassium iodide.

The oral care carrier for the oral delivery for humans include, but are not limited to, mouthwashes, toothpastes, tooth powders, prophylaxis pastes, lozenges, gums that contain a safe and effective concentration of iodine salt, such as potassium iodide according to the present disclosure.

“Safe and effective amount” as used herein, means a sufficient compound for enhancing the health of the oral cavity and preventing and/or treating tooth decay and gum disease associated with the bacteria while being safe to the hard and soft tissues of the oral cavity.

The terms “comprising,” “having,” “including,” and “containing” as used herein, mean that various additional components can be conjointly employed in the compositions of this invention as long as the iodine salt performs its intended functions.

The term “carrier” or “oral care carrier” as used herein, mean a suitable vehicle which is pharmaceutically acceptable and can be used to apply the present oral compositions to the oral cavity.

### DETAILED DESCRIPTION

The following definitions and non-limiting guidelines must be considered in reviewing the description of this invention set forth herein. The headings (such as “Background” and “Summary”) used herein are intended only for general organization of topics within the disclosure of the invention, and are not intended to limit the disclosure of the invention or any aspect thereof. In particular, subject matter disclosed in the “Background” may include aspects of technology within the scope of the invention, and may not constitute a recitation of prior art. Subject matter disclosed in the “Summary” is not an exhaustive or complete disclosure of the entire scope of the invention or any embodiments thereof.

The citation of references herein does not constitute an admission that those references are prior art or have any relevance to the patentability of the invention disclosed herein. Any discussion of the content of references cited is intended merely to provide a general summary of assertions made by the authors of the references, and does not constitute an admission as to the accuracy of the content of such references. All references cited in the Description section of this specification are hereby incorporated by reference in their entirety.

The description and specific examples, while indicating embodiments of the invention, are intended for purposes of illustration only and are not intended to limit the scope of the invention. Moreover, recitation of multiple embodiments having stated features is not intended to exclude other embodiments having additional features, or other embodiments incorporating different combinations the stated of features.

As used herein, the words “preferred” and “preferably” refer to embodiments of the invention that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

As used herein, the term “about,” when applied to the value for a parameter of a composition or method of this invention, indicates that the calculation or the measurement of the value allows some slight imprecision without having a substantial effect on the chemical or physical attributes of the composition or method.

Exemplary embodiments of the invention will be described in detail below.

It is known to use oral care products as a carrier for delivering components to the oral cavity for providing oral benefits such as bactericidal properties. Such compositions have the advantage of providing a subject with a convenient method for maintaining oral health.

The present invention is directed to an oral composition that includes an oral care carrier for oral delivery and iodine salt. The invention is further directed to a method of treatment of an oral cavity by orally administering the oral care composition to a subject in need thereof in order to enhance the health of the oral cavity and prevent and/or treat tooth decay and gum disease associated with, among other things, bacteria.

The iodine salt is selected from the group consisting of ammonium iodate, ammonium iodide, calcium iodate, calcium iodide, iodine monochloride, iodine trichloride, magnesium iodate, magnesium iodide, potassium iodate, potassium iodide, sodium iodate, sodium iodide, zinc iodate and zinc iodide.

The amount of iodine salt used in the oral compositions can be any amount that is safe to use in the oral cavity that is capable of enhancing the health of the oral cavity and preventing and/or treating tooth decay and gum disease, and not to exceed 2 mg per day dose as recommended by the U.S. Federal and Drug Administration for daily consumption.

This amount will vary depending on the oral care carrier selected but generally is in the range of from 0.05% to about 25%, preferably from about 0.25% to about 5%.

In various embodiments of the present invention, the oral composition contains potassium iodide in the range of from 0.05% to about 25%, and preferably from about 0.25% to about 5%.

The carrier for the iodine salt can be any vehicle suitable for use in the oral cavity. Such carriers include mouthwashes, toothpastes, tooth powders, prophylaxis pastes, lozenges, gums and the like that are more fully described hereinafter. Toothpastes and mouthwashes are the preferred systems.

Toothpastes and/or mouthwashes preferably contain from about 0.05% to about 25% of the potassium iodide by weight. Toothpastes and/or mouthwashes may also contain an abrasive polishing material, sudsing agents, flavoring agents and/or sweetening agents. Toothpaste compositions additionally contain binders, humectants and water.

The oral composition of the present invention preferably includes an abrasive material. The abrasive material generally has a particle size of from about 0.1 to about 10 microns in diameter and is of various abrasive polishing materials, which do not excessively abrade teeth. Preferably, the abrasive material is highly compatible with the bactericides. These include, for example, silica xerogels such as those described in U.S. Pat. No. 3,538,230 to Pader et al, issued Nov. 3, 1970; hydrofluoric acid-treated amorphous silica abrasives such as those disclosed in U.S. Pat. No. 3,862,307 to DiGiulio, issued Jan. 21, 1975; mineral abrasives coated with cationic polymers such as those disclosed by J. J. Benedict in U.S. Pat. No. 4,157,387, issued Jun. 5, 1979; and condensation products of urea and formaldehyde such as those disclosed in Cooley et al, in U.S. Pat. No. 3,070,510, issued Dec. 24, 1972. All of these patents are incorporated herein by reference.

The oral composition of the present invention may also include emulsifying agents. Preferred emulsifying agents are those which are reasonably stable and foam throughout a wide pH range, including non-soap non-ionic, cationic, zwitterionic and amphoteric organic synthetic detergents.

Toothpastes, as a preferred embodiment, may also contain a thickening agent to provide a desirable consistency. Preferred thickening agents are carboxyvinyl polymers, hydroxyethyl cellulose and water soluble salts of cellulose ethers such as sodium carboxymethyl cellulose and sodium carboxymethyl hydroxyethyl cellulose. Natural gums such as gum karaya, gum arabic, and gum tragacanth can also be used. Colloidal magnesium aluminum silicate or finely divided silica can be used as part of the thickening agent to further improve texture. Thickening agents in an amount from 0.5% to 5.0% by weight of the total composition are generally used.

Humectant material is also can be used in a toothpaste in order to keep it from hardening. Preferred humectants include glycerin, sorbitol, and other edible polyhydric alcohols. The humectant can comprise up to about 65% by weight of the toothpaste composition.

Adding flavoring and/or sweeting agents is also desirable. Preferred flavoring agents include oil of wintergreen, oil of peppermint, oil of spearmint, oil of sassafras, and oil of clove. Sweetening agents which can be used include aspartame, acesulfame, saccharin, dextrose, levulose and sodium cyclamate. Flavoring and/or sweetening agents are generally used in at levels of from about 0.005% to about 2% by weight.

A mouthwash composition is another preferred embodiment of the present invention. According to this embodiment, the mouthwash composition contains from about 0.05% to about 25% of the potassium iodide by weight.

Mouthwashes generally comprise about 20:1 to about 2:1 of a water/ethyl alcohol solution and preferably other ingredients such as flavor, sweeteners, humectants and sudsing agents such as those mentioned above for dentifrices.

Generally, the mouthwashes of the present invention comprise ethyl alcohol, a humectant, an emulsifying agent, a sweetening agent, a flavoring agent, and the water.

Lozenge and chewing gum components are disclosed in U.S. Pat. No. 4,083,955, Apr. 11, 1978 to Grabenstetter et al, incorporated herein by reference.

Many other compounds can be used in addition to those discussed above, such as, for example anti-stain compounds, that are suitable and do not diminish the effectiveness of the potassium iodide in enhancing the health of the oral cavity and preventing and/or treating tooth decay and gum disease associated with the bacteria. Materials among those that are useful in carriers include adhesion agents, viscosity modifiers, diluents, surfactants, foam modulators, peroxide activators, peroxide stability agents, abrasives, pH modifying agents, humectants, mouth feel agents, sweeteners, flavorants, colorants, and combinations thereof. It is understood that while general attributes of each of the above categories of materials may differ, there may be some common attributes and any given material may serve multiple purposes within two or more of such categories of materials. Preferably, such carrier materials are selected for compatibility with the potassium iodide and with other ingredients of the oral composition.

Colorants may also be used with the oral composition according to the present disclosure. Colorants may include pigments, dyes, lakes and agents imparting a particular luster or reflectivity such as pearling agents. Any orally acceptable colorant can be used, including FD&C dyes and pigments, talc, mica, magnesium carbonate, calcium carbonate, magnesium silicate, magnesium aluminum silicate, silica, titanium dioxide, zinc oxide, red, yellow, brown and black iron oxides, ferric ammonium ferrocyanide, manganese violet, ultramarine, titaniated mica, bismuth oxychloride, and mixtures thereof. One or more colorants are optionally present in a total amount of about 0.001% to about 20%, for example about 0.01% to about 10% or about 0.1% to about 5%.

The pH of the oral composition and/or its pH in the mouth can be any pH which is safe for the hard and soft tissues of the mouth. Such pHs are generally from about 3 to about 10, preferably from about 4 to about 8. Examples of acceptable buffers and buffer systems include sodium bicarbonate, disodium phosphate/sodium phosphate and monobasic potassium phosphate/sodium hydroxide.

The carrier of the oral compositions of the present invention can be manufactured according to the methods and industry standards that are common in the oral care industry. For example, the oral compositions of the present invention can be made by adding and mixing the ingredients of the oral composition in a suitable vessel such as a stainless steel tank provided with a mixer.

The method, according to the embodiment of the present invention, involves treatment of an oral cavity by orally administering the oral care composition to a subject in need thereof. Generally an amount of at least about 0.05% by weight of potassium iodide is effective.

All percentages, parts, and ratios are calculated by weight unless otherwise indicated. All percentages and ratios are calculated based on the total composition unless otherwise indicated.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

Uses of singular terms such as “a,” “an,” are intended to cover both the singular and the plural, unless otherwise indicated herein or clearly contradicted by context. All references, including publications, patent applications, and patents, cited herein are hereby incorporated by reference. Any description of certain embodiments as “preferred” embodiments, and other recitation of embodiments, features, or ranges as being preferred, or suggestion that such are preferred, is not deemed to be limiting. The invention is deemed to encompass embodiments that are presently deemed to be less preferred and that may be described herein as such. All methods described herein can be performed in any suitable order unless otherwise indicated herein or otherwise clearly contradicted by context. The use of any and all examples, or exemplary language (e.g., “such as”) provided herein, is intended to illuminate the invention and does not pose a limitation on the scope of the invention. Any statement herein as to the nature or benefits of the invention or of the preferred embodiments is not intended to be limiting. This invention includes all modifications and equivalents of the subject matter recited herein as permitted by applicable law. Moreover, any combination of the above-described elements in all possible variations thereof is encompassed by the invention unless otherwise indicated herein or otherwise clearly contradicted by context. The description herein of any reference or patent, even if identified as “prior,” is not intended to constitute a concession that such reference or patent is available as prior art against the present invention. No unclaimed language should be deemed to limit the invention in scope. Any statements or suggestions herein that certain features constitute a component of the claimed invention are not intended to be limiting unless reflected in the appended claims.

The invention being thus described, it will be obvious that the same may be varied in many ways. Such variations are not to be regarded as a departure from the spirit and scope of the invention, and all such modifications as would be obvious to one skilled in the art are to be included within the scope of the following claims.

1. An anti-microbial oral care composition for treating and/or preventing tooth decay and gum disease caused by bacteria, fungi and/or viruses, comprising:
an oral care carrier for oral delivery; and
iodine salt,
wherein a concentration range of the iodine salt is from about 5.25% to about 25.0%,
wherein the anti-microbial oral care composition is administered to a subject in need thereof, and
wherein the anti-microbial oral care composition has a pH of 7.0 or higher.

2. The oral care composition according to claim 1, wherein the iodine salt is selected from the group consisting of ammonium iodate, ammonium iodide, calcium iodate, calcium iodide, iodine monochloride, iodine trichloride, magnesium iodate, magnesium iodide, potassium iodate, potassium iodide, sodium iodate, sodium iodide, zinc iodate and zinc iodide.

3. The oral care composition according to claim 1, wherein the iodine salt is potassium iodide.

4. (canceled)

5. The oral care composition according to claim 1, wherein the oral care carrier for oral delivery is oral rinse, mouthwash or toothpaste.

6. A method of treatment of oral cavity for preventing and/or treating tooth decay and gum disease caused by bacteria, fungi and/or viruses comprising:
orally administering to a subject in need thereof an anti-microbial oral care composition,
wherein the anti-microbial oral care composition comprises:
an oral care carrier for oral delivery; and
iodine salt,
wherein a concentration range of the iodine salt is from about 5-% 5.25% to about 25.0%, and
wherein the anti-microbial oral care composition has a pH of 7.0 or higher.

7. The method according to claim 6, wherein the iodine salt is selected from the group consisting of ammonium iodate, ammonium iodide, calcium iodate, calcium iodide, iodine monochloride, iodine trichloride, magnesium iodate, magnesium iodide, potassium iodate, potassium iodide, sodium iodate, sodium iodide, zinc iodate and zinc iodide.

8. The method according to claim 6, wherein the iodine salt is potassium iodide.

9. (canceled)

10. The method according to claim 6, wherein the oral care carrier for oral delivery is mouthwash or toothpaste.
